(19) United States
(12) Patent Application Publication
(10) Pub. No.: US 2002/0006650 A1
(43) Pub. Date: Jan. 17, 2002

(54) SOLID CULTURE SUBSTRATE INCLUDING BARLEY

(76) Inventors: CLIFFORD A. BRADLEY, BUTTE, MT (US); PAULINE P. WOOD, BUTTE, MT (US); WILLIAM E. BLACK, BUTTE, MT (US); ROBERT D. KEARNS, BUTTE, MT (US); JAMES BRITTON, BUTTE, MT (US)

Correspondence Address: LAHIVE & COCKFIELD, LLP, 28 STATE STREET, BOSTON, MA, 02109

(21) Appl. No.: 09/291,412

(22) Filed: Apr. 13, 1999

Related U.S. Application Data
(63) Continuation of application No. 08/430,296, filed on Apr. 28, 1995, now abandoned.
(63) Continuation of application No. 08/134,849, filed on Oct. 12, 1993, now abandoned.

(51) Int. Cl. C12N 11/00; C12N 11/16; C12N 5/00

(52) U.S. Cl. 435/174; 435/395

(57) ABSTRACT

Solid culture barley substrates for growing fungus are described. Solid cultures, packaged solid culture substrates, and methods for growing fungus using the solid culture substrates also are described.

### RELATED APPLICATIONS

This application is related to applications entitled “Formulations of Entomopathogenic Fungi for Use as Biological Insecticides” and “Mycoinsecticides Against an Insect of the Grasshopper Family”, filed on even date herewith, the contents of each of the aforementioned applications are hereby expressly incorporated.

### BACKGROUND OF THE INVENTION

A wide variety of techniques are available for growing fungi including submerged (liquid) culture, surface culture, two-step processes and solid substrate culture. In submerged culture, the fungus is grown in a liquid media. The main disadvantage of submerged culture is that the fungi typically produce blastospores rather than “true” conidia with a hardened cell wall. Blastospores are unstable with a limited shelf life and poor field stability.

In surface culture, the fungus is grown and conidiates on the surface of a liquid or solid medium. With respect to liquid surface cultures, a shallow layer of media is inoculated with a fungal culture which grows as a mat on the liquid surface.

Two-step processes combine conventional submerged culture with production of stable aerial conidia. In both of the two common two-step processes, mycelia are initially grown in liquid culture. In one process, the mycelia are then applied to a solid, absorbent material where the fungus conidiates as a surface culture. In the other process, the mycelia are dried and milled to produce cell fragments that conidiate after application in the field.

Solid substrate culture is a widely used production technique. In solid substrate culture, the fungus grows on the surface of a moist solid substrate. Commonly used solid substrates include agar, rice, wheat, corn, millet and corn starch.

High conidia yield is essential to economical production of a fungus. In solid substrate culture, economic efficiency improves with increasing depth of culture substrate bed, the surface area that can be obtained in a given volume, and the number of conidia obtained per unit weight of substrate used in the culture. To achieve depth and surface area, the physical characteristics of the substrate are important. As bed depth increases, it becomes more difficult to achieve uniform air flow, temperature control, and moisture control as weight compresses the bed and starch is used by the fungus.

### SUMMARY OF THE INVENTION

The present invention is based, at least in part, on the discovery that barley can be used as a solid culture substrate for growing fungi. Barley has nutritional characteristics that support equal or superior conidia production compared with other grains typically used in solid culture, e.g. rice. Barley also has good physical characteristics (either alone or in combination with an inert material or solid support) for solid culture. Barley's physical characteristics are superior to those of rice which is a commonly used grain for solid culture. Appropriate bed depths and surface areas can be achieved with barley allowing for its use in a mass production process for growing a fungus. Barley can be used alone as a solid substrate, as in processed barley, or in combination with an inert material or solid support. The present invention provides solid culture substrates which include barley in a form suitable for growing an entomopathogenic fungus. The invention also provides solid culture substrates which include processed barley in a form suitable for growing a fungus.

The present invention also provides solid cultures which include barley and a culture of a fungus. The fungus can be an entomopathogenic fungus and the barley can be processed barley.

Other aspects of the present invention include methods of growing a fungus, e.g. an entomopathogenic fungus, that include combining a barley substrate (processed or unprocessed) and a culture of the fungus. The substrate and fungus are combined under conditions which support the growth of the fungus. A packaged solid culture substrate that includes a barley substrate and instructions on how to use the barley substrate to grow fungi also are part of this invention.

### DETAILED DESCRIPTION OF THE INVENTION

The present invention pertains to a solid culture substrate for growing entomopathogenic fungi. The substrate is barley in a form which is suitable for growing entomopathogenic fungi. Another embodiment of the present invention is a solid culture substrate of processed barley in a form suitable for growing a fungus.

The language “solid culture” is art-recognized and is intended to include forms of cultivating fungi on solid substrates, e.g., barley flakes, pearled barley, rice or wheat bran. Solid culture also includes methods of cultivating fungi on inert materials which have been coated with an appropriate substrate such as finely ground grain.

The term “substrate” is intended to include a surface or medium on which fungus lives or grows. Substrates are used in the production of fungal cultures to promote growth and conidiation of the fungus. Examples of substrates within the scope of this invention include pearled barley, ground barley, coated plastic rings or corn cob woody fractions, and water extracts of barley sorbed onto diatomaceous earth.

The language “in a form suitable for growing an entomopathogenic fungus” is intended to include forms of the solid culture substrate upon which an entomopathogenic fungus can be grown or produced. The form of the solid culture substrate can be determined by the size and shape of the barley itself or by the size and shape of the inert material that the barley is coated or sorbed on. Factors considered when designing an appropriate form of the solid culture substrate include desired physical strength (compression resistance) of the substrate, void space, amount of barley per volume of substrate if attached to a solid support, and surface area to volume ratio. Suitable forms of barley include pearled barley, barley flakes, ground barley coated on inert materials, e.g., corn cob woody fractions, and water extracts of barley sorbed onto solid supports, e.g., diatomaceous earth.

The term “fungus” is art-recognized. Fungi included in the present invention are entomopathogenic fungi and fungi which can produce commercially useful enzymes such as amylase and/or ligninase. Examples of fungi include fungi of the subdivision Deuteromycotina (or Deuteromycetes) and fungi of the class Hyphomycetes. Generally, fungi of the class Hyphomycetes can produce conidia. Examples of entomopathogenic fungus genera include Beauveria, Metarhizium, Paecilomyces, Tolypocladium, Aspergillus, Culicinonyces, Nomuraea, Sorosporella, Verticillium, and Hirsutella. Examples of fungus genera that are not entomopathogenic include Trichoderma and Alternaria. Examples of species of fungus include Beauveria bassiana, Metarhizium flavoviride, Metarhizium anisopliae, Paecilomyces fumusoroseus, Paecilomyces farinosus, Nomuraea rileyii, Aspergillus niger, Aspergillus awamori, Trichoderma riride, and Trichoderma harzianium.

The language “entomopathogenic fungus” means a fungus which is capable of killing an insect. Such a fungus is considered a mycopesticide. Entomopathogenic fungi include those strains or isolates of fungal species in the class Hyphomycetes which possess characteristics allowing them to be virulent against insects. These characteristics include formation of stable infective conidia. An effective entomopathogenic fungus preferably is lethal for target insects but less harmful for non-target insects at the lethal dose for the target insects. Also, the entomopathogenic fungus preferably does not harm vegetation or animals who might come in contact with it.

The language “processed barley” is intended to include forms of barley that are not in their natural or raw state. Processed barley includes barley grain that has been changed or processed since removal from the barley plant. An example of processed barley is barley flakes, barley that has been pearled to remove the hull and then steam rolled. “Pot barley” is not intended to be processed barley for purposes of this invention.

The term “barley” is art-recognized. Barley typically includes plants which are of the genus Hordeum. Very high yields of conidia can be obtained when entomopathogenic fungi are grown in solid culture using barley as a substrate. Barley can be used in a number of forms—processed barley alone, barley mixed with other materials such as straw, finely ground barley coated onto inert carriers such as plastic rings, etc. In addition to nutritional characteristics that support equal or superior conidia production compared with other grains, barley has good physical characteristics for solid culture. Barley flakes can be pearled to remove the hull and then steam rolled. Because of the size and shape of the flakes, packed beds can be aerated and fungal growth and conidia production can occur through the entire bed. Barley flakes have good water sorption characteristic—up to 75% moisture content. Barley flakes also hold their shape and physical particle integrity. By comparison, rice, a commonly used grain for solid culture, has poor characteristics. Rice grain packs very densely which inhibits air flow and physical space for culture growth. Rice also has poor water sorption capacities and is “mushy” at a water content needed for good fungal growth, e.g. 45-55%.

The invention further pertains to a solid culture substrate for growing Deuteromycete fungi. The language “Deuteromycete fungus” is intended to include fungi that are recognized in the art as being Deuteromycete fungi. Deuteromycete fungi are fungi of the subdivision Deuteromycotina. There are two classes of Deuteromycete—Hyphomycetes and Coelomycetes. Hyphomycetes fungi generally produce conidia. Deuteromycete fungi include fungi of the genera Beauveria, Metarhizium, Paecilomyces, Tolypocladium, Aspergillus, Culicinonyces, Nomuraea, Sorosporella, and Hirsutella.

Another embodiment of this invention is a solid culture which includes barley as a substrate and a culture of an entomopathogenic fungus. The term “culture” is intended to include the environment created for the cultivation of fungi. Culture also includes inoculum cultures which are used to inoculate the substrate in the production of conidia of fungi. A further embodiment is a solid culture which includes processed barley as a substrate and a culture of fungus.

Another embodiment of this invention is a solid culture substrate that includes ground barley coated onto an inert solid support. The language “ground barley” is intended to include barley which has been reduced to particles by grinding. Grinding the barley grains increases the surface area available to the growing fungus and allows more of the barley to be utilized by the fungus, thereby, improving the economics of growing entomopathogenic fungi. Ground barley includes finely or coarsely ground barley.

Finely ground barley can be used to coat inert materials of different sizes and configurations. The size and configuration of the inert material is varied to achieve different levels of physical strength (compression resistance), void space, amount of barley per volume of substrate, and surface area to volume ratio. This approach has been used to obtain very high conidia yields per weight of barley in the culture. For example, bed depths of up to two meters and barley utilization of up to eighty percent have been achieved using substrates of barley coated onto plastic “rings”. These rings are open cylinders used in distillation commonly referred to as pull rings. Similarly, the woody fractions of corn cobs can be used as the inert material with comparable results.

The term “coated” is intended to include covering at least a portion of a first material with a layer of another substance. Preferably, the entire inert material is coated with the barley. Preferably, coated includes wetting the material with a liquid which causes the substance to stick to the material and remain there after the liquid has evaporated. The liquid can be plain water, a nutrient solution, or any solution which is not detrimental to growing conidia of an entomopathogenic fungus.

The term “inert” is art-recognized and is intended to include materials capable of providing support for the fungi without detrimentally effecting the growth of fungi. The language “nutritionally inert” is intended to include materials that do not provide nourishment to fungi. Nutritionally inert materials typically do not affect the growth of an entomopathogenic fungus either positively or negatively. Nutritionally inert materials include corn cob woody fractions, plastic rings, diatomaceous earth in a range of particle sizes and forms, ceramic rings, ceramic saddles, stainless steel rings (i.e., pull rings), crystalline silica in various forms (i.e., beads or extruded shapes), and clay of various types and forms (i.e., attaclay or kaolin formed into beads or other shapes). The particle size and form of the inert support can be selected based on such factors as the desired bed depth, desired surface area and other desired features as discussed above.

The language “solid support” is intended to include solid materials that can serve as a foundation for a substance. Solid support materials include materials that can be coated with ground barley or can absorb a barley extract without affecting the properties of the barley or the growth of an entomopathogenic fungus. Solid supports include corn cob woody fractions, plastic rings, diatomaceous earth in any of a range of particle sizes and forms, ceramic rings, ceramic saddles, stainless steel rings (i.e., pull rings), crystalline silica in various forms (i.e., beads or extruded shapes), and clay of various types and forms (i.e., attaclay or kaolin formed into beads or other shapes).

Another embodiment of the present invention is a solid culture substrate which includes barley extracts sorbed onto an inert support. For example, water barley extracts can be sorbed onto diatomaceous earth.

The language “barley extract” is intended to include a solid, viscous, or liquid substance containing the essence or the substance of barley. A barley extract can be in diluted or concentrated form. Barley extracts include water extracts of barley. For example, a barley extract can be obtained by boiling barley flakes in salt media. The resulting solution is then filtered. A method used to obtain a barley extract is described in detail in Example 5 below.

The language “sorbed onto an inert solid support” is intended to include a substance gathered on a solid support. The gathering can be by absorption, adsorption, or a combination of the two processes. A liquid can be sorbed onto a material when the liquid is mixed with the sorbent material under conditions which favor absorption, adsorption or both. For example, a barley extract can be sorbed onto a porous material such as diatomaceous earth. The method used to sorb barley extract onto diatomaceous earth is described in detail in Example 5 below.

Yet another embodiment of the invention is a solid culture substrate that includes barley mixed with an inert material. The language “inert material” is intended to include material which is non-reactive and does not detrimentally effect the growth of fungi. The inert material can be used, for example, as a bulking agent to increase the void space of the culture bed. Inert material includes straw (any grain), sawdust, wood chips, corn cob fractions, grain hulls, plastic rings, diatomaceous earth in any of a range of particle sizes and forms, ceramic rings, ceramic saddles, stainless steel rings (i.e., pull rings), crystalline silica in various forms (i.e., beads or extruded shapes), and clay of various types and forms (i.e., attaclay or kaolin formed into beads or other shapes).

Another embodiment of the invention is a packaged solid culture substrate. The packaged solid culture substrate includes barley as a substrate and instructions for using the barley to grow entomopathogenic fungi. In addition, the present invention provides a packaged solid substrate that includes processed barley as a substrate and instructions for using the processed barley to grow fungi. The barley can take forms including barley flakes, pearled barley, barley extract or ground barley.

The term “instructions” is intended to include knowledge or information imparted to a person or persons in printed form regarding the proper usage of barley as a substrate for growing fungi. The instructions further can include information on how to use the barley in combination with an inert material or a nutritionally inert solid substrate to achieve desired physical characteristics.

Other embodiments of the present invention include methods of growing an entomopathogenic fungus on the solid culture substrate described above. The method includes providing a barley substrate and then combining the barley substrate with a culture of an entomopathogenic fungus. The combination is made under conditions suitable for growth of entomopathogenic fungi. Examples of these conditions are described in detail in the examples below. Conidia can be recovered from the combination after an appropriate period of time using conventional techniques, e.g. passing through a mill. Similarly, another embodiment of the present invention is a method for growing a fungus on processed barley using the same steps as described above.

The language “barley substrate” is as defined above. The term “conidia” is art-recognized and is intended to include asexual spores characteristic of many fungi including entomopathogenic fungus. Conidia generally have a hardened cell wall. However, conidia also includes blastospores which do not have hardened cell walls. Conidia of a fungus can be counted by standard techniques and used as units of measure of the fungus, for example, to determine the total number of conidia per unit weight or volume and as a basis for determining viability or bioactivity, e.g., LD₅₀ in an insect.

The language “significantly higher” is intended to include a level of conidia yield that is art-recognized as being significantly higher when compared to another level of conidia yield after a selected period of time. Significantly higher amounts are at least about 10% higher, preferably at least about 25% higher, more preferably at least about 50% higher and most preferably at least about 100% or more higher.

The methods of the present invention also include the growth of light-sensitive or light-dependent fungi. Light-sensitive or light-dependent fungi are fungi which typically require light for forming conidia in culture. The barley substrate of the present invention alleviates or substantially reduces the effect of the light requirement for conidiation of a light-dependent fungus. The alleviation of the light requirement reduces the expenses for culturing. An example of a fungi dependent on light for conidia formation is Paecilomyces fumusoroseus.

The invention is further illustrated by the following non-limiting examples. The contents of all cited copending applications, issued patents, and published references are hereby expressly incorporated by reference.

### EXAMPLES

### Example 1

### A Comparison of Conidia Yield of Entomopathogenic Fungi Grown on Different Grains

In this experiment, conidia production of three different insect pathogenic fungi grown on seven different grains was evaluated. This experiment was conducted with milled grain incorporated into agar in a petri dish so that variations in physical characteristics of the different grains in solid culture was not a factor in conidia yields.

Fungi were Beauveria bassiana strain GHA1991 used against grasshoppers, Beauveria bassiana strain GMB6 originally isolated from the gypsy moth and Metarhizium anisopliae ARSEF 2134, isolated from the Japanese beetle.

Seven different grains were obtained from commercial sources; barley, spring wheat, winter wheat, oats, rye, millet and rice grains were fine ground. Resulting grain flour was mixed at 3% w/w with water, 1.5% agar added, autoclaved for 20 minutes at 15 psi, 121° C. and poured into 15 cm diameter petri dishes to about 5 mm deep.

Inoculum cultures of GMB6 and Metarhizium anisopliae were prepared by inoculating broth cultures from maintenance slants. Broth was CSYE broth containing

40 g/l glucose

10 g/l KNO₃

5 g/l KH₂PO₄

1 g/l MgSO₄

0.05 g/l CaCl₂

2 g/l yeast extract

Inoculum cultures were grown for three days at 25° C. in a rotary shaking water bath.

GHA 1991 inoculum was grown in a broth medium composed of:

1 g/l KH₂PO₄

1 g/l yeast extract

10 g/l molasses

pH to 3.8 with H₂SO₄

Inoculum for plates was from the second transfer of broth culture to fresh broth with a final volume of 100 liters.

Beauveria bassiana inoculum cultures contained 2×10⁸ to 3×10⁸ blastospores per ml and Metarhizium anisopliae inoculum contained 1.2×10⁶ blastospores per ml. Beauveria bassiana inoculum was diluted and applied to plates at the rate of 1×10⁶ blastospores per plate. Metarhizium anisopliae inoculum was applied at 4.8×10⁵ blastospores per plate.

Plates were incubated at 25° C. for ten days in the dark. Plates were covered by a uniform surface culture of fungus. Conidia production was monitored by taking 0.5 cm² plugs from the plates and homogenizing the plugs in a premeasured volume of 0.1% Tween 80 to disperse conidia. Conidia in the homogenate were diluted as appropriate and were counted in a hemocytometer at 400× magnification in a phase contrast microscope. Results are expressed as number of conidia per 0.5 cm² plug. Duplicate plates of each grain and strain were made and three plugs sampled from each plate for a total of six samples for each strain and grain combination. Results are expressed as the average of these six samples. The GMB6 plates were run about one week prior to the Metarhizium anisopliae and GHA plates.

TABLE 1
Conidia Yields of Entomopathogenic Fungi
Grown on Different Grains
Conidia per 0.5 cm diameter Agar Plug

| Grain | BbGMB6 | BbGHA1991 | M. anisopliae |
|---|---|---|---|
| Barley | 3.05 × 10⁸ | 2.84 × 10⁸ | 1.97 × 10⁷ |
| Wheat | | | |
| Spring | 1.36 × 10⁸ | 2.02 × 10⁸ | 3.33 × 10⁷ |
| Winter | 1.36 × 10⁸ | 1.84 × 10⁸ | 3.29 × 10⁷ |
| Oats | 2.21 × 10⁸ | 1.25 × 10⁸ | 3.97 × 10⁷ |
| Rye | 2.50 × 10⁸ | 1.54 × 10⁸ | 3.27 × 10⁷ |
| Millet | 7.58 × 10⁷ | 2.14 × 10⁸ | 6.73 × 10⁶ |
| Rice | 9.65 × 10⁷ | 4.16 × 10⁷ | 1.58 × 10⁷ |

The highest conidia counts for Beauveria bassiana were on barley. For Metarhizium flavoviride, the highest conidia yields were on oats. However, wheat and rye gave similar results for Metarhizium flavoviride. A commonly used substrate for solid culture of entomopathogenic fungi is rice. The yields for all three fungi on barley were greater than on rice.

### Example 2

### Conidia Production of Beauveria bassiana Strain BbGHA1991

The strain was maintained as a dried laboratory solid culture stored at 4° C. Broth cultures of the composition described below were inoculated with conidia from this maintenance culture and incubated at 25° C. on a rotary shaking water bath for three to six days. Broth culture medium results in production of high numbers of single-celled blastospores. Typical broth cultures contain in excess of 1×10⁸ blastospores per ml. For some larger cultures, 100 ml broth culture were transferred to 1.5 liters of broth in 2800 ml flasks and incubated at 25° C. with approximately 500 cc/minute sparged air flow.

Inoculum Culture Medium

| | |
|---|---|
| Glucose | 40 g/l |
| KNO₃ | 10 g/l |
| KH₂PO₄ | 5 g/l |
| MgSO₄ | 1 g/l |
| CaCl₂ | 0.05 g/l |
| Yeast extract | 2 g/l |

Solid culture substrate was prepared by mixing equal parts by weight of dry barley flakes and one culture medium described above except that glucose was omitted. Wetted barley was autoclaved in polypropylene bags for 20 minutes to one hour (depending on volume) at 15 psi, 121° C., cooled and inoculated by transferring broth cultures directly to bags of substrate which were mixed by hand. Generally 1.5 kg dry weight of barley flakes were mixed with 1500 ml nutrient solution and autoclaved in one bag, cooled and inoculated with 300 ml of broth inoculum culture. Inoculated solid substrate was transferred to an autoclave-sterilized, polycarbonate box 27 cm×48 cm ×15 cm fitted with a screen bottom and connectors for air inlet and outlet. The substrate formed a bed about eight to ten centimeters deep on the screen. In some cultures, 3 kg dry weight of flakes were processed and incubated in 18″ diameter×24″ deep round steel vessels fitted with screens. The culture beds were about 20 to 30 cm deep. Cultures were incubated at 20-30° C. for 10 days with an air flow of about 0.5 to 2 liters/minute. Air flow was varied to maintain culture temperature. After eight to twelve days incubation, cultures were transferred to a dryer consisting of screens inside and equipped with a fan. Culture was spread on screens and dried to a final moisture content of less than 10% with a flow of dry air at 20-25° C.

Dried cultures were passed through a Wiley mill which had cutting blades removed. This removed conidia from dried barley flakes by turbulence and particle-to-particle abrasion without significantly reducing the size of residual barley flakes. The mill discharged to a covered vibrating 80 mg mesh (US Standard) screen (Sweco Model LS1885333) fitted with a cover. Material that passed the screen was weighed and assayed for concentration of viable conidia by methods described below:

0.1 g conidia preparation was weighted into 9.9 ml

0.1% Tween 80 solution in a Potter-Elvejhem homogenizer tube

Conidia suspension was homogenized for two minutes and diluted as appropriate (generally diluted to contain an estimated 1×10⁶ to 1×10⁷ conidia/ml in the final dilution.) Conidia concentration was determined by microscopic count at 400× magnification using a hemocytometer. (Neubauer-Levy or Petroff-Hauser Chamber or equivalent). Viability was determined by placing a drop of diluted conidia suspension on Sabaroud's Dextrose Agar Yeast Extract (SDAY, Difco) plate.

The drop was covered with a sterile microscope cover slip and plates were incubated 16-20 hours at 25° C. Plates were examined at 400× and germinated and ungerminated conidia were each counted. Conidia were considered germinated if swollen or if a hypha was emerging from the conidia. Conidia suspensions were sampled in duplicate. For each sample, a total of at least 100 conidia were counted in at least three microscope fields of view.

Table 3 summarizes results from 56 cultures with 1.5, 2 or 3 kg dry weight substrate input run in seven separate sets over about a 2½ month period. Table 2 shows substrate input, recovered conidia powder, total recovered conidia and calculates conidia yield and concentration. Conidia yield averaged 5.7×10¹² per kg substrate input with an average concentration of 7.6×10¹⁰ conidia/gram.

Milling and screening recovered approximately one-half of the conidia produced in the culture. Residual material from a number of cultures was remilled and screened with recovery of a quantity of conidia almost equal to the recovery in the first pass. Counts of conidia remaining on residual culture material after milling and screening also showed considerable conidia concentration, typically on the order of 10⁹ per gram. Based on these observations, total conidia production exceeds 1×10¹³ per kg of substrate input.

TABLE 2

| Totals for seven sets were: | |
|---|---|
| Total substrate input: | 91 kg |
| Total Weight recovered spore powder: | 6823 g |
| Total conidia recovered: | 5.17 × 10¹⁴ |
| Average Concentration conidia: | 7.6 × 10¹⁰ |
| Average yield per kg substrate input: | 5.7 × 10¹² conidia |

TABLE 3
Conidia Production B bassiana GHA1991

| | | Set 1 | Set 2 | Set 3 | Set 4 | Set 5 | Set 6 | Set 7 | Total |
|---|---|---|---|---|---|---|---|---|---|
| 1) | Number of Cultures | 14 | 10 | 5 | 8 | 6 | 6 | 7 | 56 |
| 2) | Total Substitute Input | 25 | 16.5 | 9.0 | 12.0 | 9.0 | 9.0 | 10.5 | 91 |
| | (kg dry wt) | | | | | | | | |
| 3) | Total Recovered | 1964 | 1107 | 703 | 733 | 682 | 809 | 809 | 6807 |
| | Conidia powder (g) | | | | | | | | |
| 4) | Total Conidia | 1.47 × 10¹⁴ | 7.17 × 10¹³ | 6.48 × 10¹³ | 5.2 × 10¹³ | 5.95 × 10¹³ | 5.75 × 10¹³ | 6.52 × 10¹³ | 5.18 × 10¹⁴ |
| | Recovered | | | | | | | | |
| 5) | Average Conidia | 7.5 × 10¹⁰ | 6.4 × 10¹⁰ | 9.2 × 10¹⁰ | 7 × 10¹⁰ | 8.7 × 10¹⁰ | 7.1 × 10¹⁰ | 7.9 × 10¹⁰ | 7.6 × 10¹⁰ |
| | Concentration Conidia | | | | | | | | |
| | per Gram of Powder | | | | | | | | |
| 6) | Average Conidia Yield | 5.9 × 10¹² | 4.3 × 10¹² | 7 × 10¹² | 4.3 × 10¹² | 6.6 × 10¹² | 6.4 × 10¹² | 6.2 × 10¹² | 5.7 × 10¹² |
| | Conidia per kg | | | | | | | | |
| | Substrate Input | | | | | | | | |

### Example 3

### Production of Metarhizium Flavoviride Conidia on Barley Substrate

Metarhizium flavoviride USDA ARSEF 7023 was grown in laboratory solid culture on barley flakes. Slant cultures were used to inoculate CSYE broth which were then grown for three days at 28° C. Broth cultures were used to inoculate barley flake substrate prepared as follows. 50 grams of barley flakes were wetted with 50 ml salt solution, autoclaved and cooled. Then 10 ml inoculum culture was added, mixed, and the inoculated substrate was transferred to 200 cc columns which were then incubated at 25° C. with 10-15 cc/minute humidified air flow for ten days. After ten days, the cultures were transferred to a vacuum oven and dried. The dried cultures were ground, screened through 60 mesh and the recovered conidia powder weighed and assayed for concentration of viable conidia by procedures described in Example 2.

Duplicate cultures gave the following yields:

| | |
|---|---|
| Culture 1: | 6.7 g powder |
| | 8.6 × 10¹⁰ conidia/gram |
| | 95% viable |
| Culture 2: | 6.Og powder |
| | 8.9 × 10¹⁰ conidia/gram |
| | 95% viable |
| Total: | 12.7 g powder |
| | 10 |
| | 8.7 × 10¹⁰ conidia/gram |

The yield is equivalent to 1.1×10¹³ conidia per kg dry weight of barley flakes. These yields were similar to yields of Beauveria bassiana.

### Example 4

### Beauveria Bassiana Grown on Diatomaceous Earth Particles With Barley

Beauveria bassiana ARSEF 252 was grown on substrates composed of barley with diatomaceous earth (DE) as an inert support. Two types of substrates were prepared: 1) DE coated with barley flour and 2) DE with barley extract sorbed into the DE.

Beauveria bassiana inoculum culture was grown from maintenance slants in CSYE broth.

Diatomaceous earth was obtained from Eagle Pitcher, Reno, Nev., sold under the trade name, Celatom. Three sizes were used:

MP-94 Coarse (4-6 mesh)

MP-77 Medium variable size (8-80 mesh)

MP-99 Medium one size (6-10 mesh).

Barley-coated-DE was prepared by mixing 5 g barley flour in 100 ml salt media and mixing with 85 g DE. Salt media used was:

10 g/l KNO₃

5 g/l KH₂PO₄

2 g/l MgSO₄

0.05 g/l CaCl₂

Barley extract-DE was prepared by boiling 50 g barley flour in one liter of salt media for two to three minutes, then filtering through five layers of cheese cloth. 100 ml of the extract was added to 85 gDE.

DE substrates were autoclaved, cooled, inoculated with 15 ml inoculum culture and transferred to 200 cc laboratory columns which were incubated at 25° C. for ten days with 5-10 cc/minute humidified air flow.

For comparison a DE substrate with a molasses-based medium was prepared with 100 ml of the following media added to 85 g DE and processed as above. Molasses media was:

5 g/l Molasses (Sugar beet)

1 g/l KH₂PO₄

1 g/l Yeast extract, pH 3.8

This molasses media is used for Beauveria bassiana broth cultures and supports blastospore production in liquid inoculum culture of greater than 1×10⁸ /ml. At ten days, barley DE substrates showed extensive growth and conidiation. Molasses media showed poor growth and no visible conidiation and were not further processed.

Barley-DE cultures were dried in ambient temperature air flow. Dried culture was weighed and placed on a 60 mesh screen and shaker. Conidia powder passing through the screen and was collected, weighed and assayed for concentration of viable conidia (Table 4).

TABLE 4

| | Dry Weight | Recovered |
|---|---|---|
| | Whole Culture (g) | Conidia Powder (g) |
| DE/Barley Flour | | |
| MP99 | 100 | 2 |
| 94 | 91 | 2 |
| 77 | 77 | 2 |
| DE/Barley Extract | | |
| MP99 | 91 | 1 |
| 94 | 86 | 2 |
| 77 | 95 | 2 |

Conidia concentration (99% viability) in recovered powders

Pooled Barley extract 2.0×10¹¹/gram

Pooled Barley flour 2.2×10¹¹/gram

### Example 5

### Production of Beauveria bassiana Conidia on Barley Flour-Coated Corn Cob

A strain of Beauveria bassiana isolated from cornroot worm was grown on a woody fraction of corn cob coated with barley flour. Inoculum culture was prepared in CSYE broth from maintenance slants.

A woody fraction of corn cob was used as an absorbent, but nutritionally inert support for barley flour. The cob fraction was designated aspirated cob fraction and was obtained from Mt. Pulaski Mt. Pulaski, Ill. The cob fraction was composed of irregular, woody particles about ⅛-¼″ diameter. Barley flour was made from pearled barley and was obtained from Minnesota Grain Pearling, Cannon Falls, Minn.

1.5 kg dry cob particles were wetted with 1.5 liters of a nutrient solution consisting of: 1 g/l KH₂PO₄ and 0.4 g/l yeast extract acidified to pH 3 with H₂SO₄.

The cob pieces were allowed to soak up the nutrient solution for about five minutes at which point 375 grams of barley flour was added and thoroughly mixed. The flour stuck to and coated the cob particles. This mixture was placed in an autoclave bag and autoclaved (two bags per autoclave run) for 1.5 hours at 121° C., 15 psi, cooled and inoculated with 400 ml inoculum culture, then mixed in the autoclave bag. Autoclaved substrate was transferred to sterilized 15 cm×49 cm×29 cm polycarbonate boxes fitted with air connections. A solid culture bed, about 6 cm deep was formed. The cultures were incubated at 20-30° C. with 0.5 to 2 liters per minute humidified air flow for 8-12 days. Five replicated cultures were produced by this procedure and assayed for concentration of conidia in the whole dry culture. Five grams of culture was washed with shaking for 15 minutes in 50 ml Tween 80 solution (0.05%) and counted in a hemocytometer (Table 5). A culture was produced on barley flakes by the standard procedure as described above for comparison purposes.

TABLE 5
Production of Beauveria bassiana Conidia
on Barley Flour-Coated Corn Cob

| Corn Cob | Conidia per gram |
|---|---|
| Culture | Dry weight culture |
| 1 | 6.2 × 10⁹ |
| 2 | 5.0 × 10⁹ |
| 3 | 4.1 × 10⁹ |
| 4 | 5.5 × 10⁹ |
| 5 | 4.2 × 10⁹ |
| Flakes | 1.4 × 10¹⁰ |

Barley flakes provided a higher yield per weight of culture. However, the coated cob method used much less barley. Coated cob pieces were 25% w/w barley so that the number of conidia produced per unit weight of barley input to cultures is greater. In addition, cob pieces can be washed and recycled.

### Example 6

### Conidia Production of Paecilomyces fumusoroseus Grown on Barley

Paecilomyces fumusoroseus strain ARSEF 3572 (USDA ARS Entomopathogenic Fungi Collection) isolated from white fly (Bemesia tabaci) was grown in barley agar plates in comparison with standard laboratory media described for this species. Conidia formation in this species has also been observed to be dependent on exposure to light.

Uniform exposure of culture to light in mass production is difficult and it would be a particular advantage if a nutrient substrate were to support conidiation of fungal strains which would otherwise require light exposure.

Media

Three different agar media were tested, Sabaroud's Maltose Yeast Extract (SMAY), Sabaroud's Dextrose Yeast Extract (SDAY) and Barley Flour Yeast Agar (BFYA). SMAY and SDAY consist of nepeptone (Difco) 10 g/l, yeast extract 10 g/l and either glucose or maltose at 40 g/l. These agars are routinely used for laboratory culture of entomopathogenic fungi including Paecilomyces fumusoroseus. BFYA consists of barley flour and 10 g/l yeast extract. Two concentrations of barley flour were tested: 30 g/l and 50 g/l. All agars contained 15 g/l agar and were poured about 5 mm deep in standard 15 cm petri dishes.

Inoculum Cultures

Cultures were maintained on SDAY agar slants stored at 4° C. Inoculum cultures were prepared by transferring conidia from maintenance cultures to Sabaroud's Maltose Yeast extract broth. SMY broth is the same as above (without agar). Agar plates prepared with different media described above were inoculated with 34,000 colony-forming units per plate, spread uniformly over the agar surface. Sets of duplicate plates of each agar were inoculated and incubated in either constant light or constant dark. Light source was two General Electric® day light F400 bulbs set 12 inches above the table with petri dishes. Dark plates were wrapped in aluminum foil. Plates were incubated for 10 days at 25-27° C.

At 10 days conidia formation on each plate was assayed. Three 1.15 cm discs were cut from each plate with a cork borer, washed in 10 ml 0.10% Tween 80 and conidia in the wash suspension were counted by hemocytometer. Table 6 shows conidia per cm² from each agar incubated in the light or dark. Conidia counts are the average of six samples-three discs cut per plate, duplicated plates of each agar in light or dark conditions.

TABLE 6
Paecilomyces fumusoroseus Conidia Yields
on Different Media Under Light or Dark Incubation

| Agar medium | | Conidia/cm² | |
|---|---|---|---|
| SMAY | Light | 3.3 × 10⁸ | |
| | Dark | 1.4 × 10⁶ | 236 |
| SDAY | Light | 7.8 × 10⁸ | |
| | Dark | 2.1 × 10⁵ | 3714 |
| BFAY | Light | 2.4 × 10⁸ | |
| 30 g/l Barley Flour | Dark | 7.4 × 10⁶ | 32 |
| BFAY | Light | 4.17 × 10⁸ | |
| 50 g/l Barley Flour | Dark | 4.7 × 10⁷ | 9 |

On SMAY and SDAY light incubated plates, Paecilomyces fumusoroseus had 236 and 3700 times greater conidia concentration on light incubated versus dark incubated plates (Table 6). On barley agar plates, the effect of light was much less with a 32 fold difference between light and dark incubated plates on 30 g/l barley flour plates and 9 fold difference on 50 g/l barley flour plates (Table 6). With this species, the need for light to induce efficient conidia formation is reduced by the use of barley as the principal nutrient source. The effect of barley is concentration dependent further indicating a nutrient substrate effect in overcoming light exposure dependent conidiations.

Equivalents

Those skilled in the art will recognize, or be able to ascertain using no more than routine experimentation, numerous equivalents to the specific embodiments described herein. Such equivalents are considered to be within the scope of this invention and are covered by the following claims.

1. A solid culture substrate comprising barley in a form suitable for growing an entomopathogenic fungus.

2. The solid culture substrate of claim 1 wherein the barley is processed barley.

3. The solid culture substrate of claim 2 wherein the processed barley is selected from the group consisting of pearled barley, barley flakes, ground barley and barley flour.

4. The solid culture substrate of claim 1 wherein the entomopathogenic fungus is a Deuteromycete fungus.

5. The solid culture substrate of claim 4 wherein the entomopathogenic fungus is a Hyphomycetes fungus.

6. The solid culture substrate of claim 5 wherein the entomopathogenic fungus is a genus selected from the group consisting of Beauveria, Metarhizium, Paecilomyces, Tolypocladium, Aspergillus, and Hirsutella.

7. The solid culture substrate of claim 6 wherein the entomopathogenic fungus is a species selected from the group consisting of Beauveria bassiana, Metarhizium flavoviride, Metarhizium anisopliae, Paecilomyces fumusoroseus, and Paecilomyces farinosus.

8. A solid culture substrate comprising processed barley in a form suitable for growing a fungus.

9. The solid culture substrate of claim 8 wherein the processed barley is selected from the group consisting of pearled barley, barley flakes, ground barley and barley flour.

10. The solid culture substrate of claim 8 wherein the fungus is a Deuteromycete fungus.

11. A solid culture comprising barley as a substrate and an inoculum culture of an entomopathogenic fungus.

12. The solid culture of claim 11 wherein the barley is processed barley.

13. The solid culture of claim 12 wherein the processed barley is selected from the group consisting of barley flakes, pearled barley, ground barley, and barley flour.

14. The solid culture of claim 11 wherein the entomopathogenic fungus is a Deuteromycete fungus.

15. The solid culture substrate of claim 14 wherein the entomopathogenic fungus is a Hyphomycetes fungus.

16. The solid culture of claim 15 wherein the entomopathogenic fungus is a genus selected from the group consisting of Beauveria, Metarhizium, Paecilomyces, Tolypocladium, Aspergillus, and Hirsutella.

17. The solid culture of claim 16 wherein the entomopathogenic fungus is selected from the group consisting of Beauveria bassiana, Metarhizium flavoviride Metarhizium anisopliae, Paecilomyces fumusoroseus, and Paecilomyces farinosus.

18. A solid culture comprising processed barley as a substrate and an inoculum culture of a fungus.

19. The solid culture of claim 18 wherein the processed barley is selected from the group of barley flakes, pearled barley, ground barley, and barley flour.

20. The solid culture of claim 18 wherein the fungus is a Deuteromycete fungus.

21. A solid culture substrate comprising ground barley coated on an inert solid support

22. The solid culture substrate of claim 21 wherein the inert solid support is nutritionally inert.

23. The solid culture substrate of claim 21 wherein the solid culture substrate is in a form suitable for growing an entomopathogenic fungus.

24. The solid culture substrate of claim 21 wherein the entomopathogenic fungus is a Deuteromycete fungus.

25. The solid culture substrate of claim 24 wherein the entomopathogenic fungus is a Hyphomycetes fungus.

26. The solid culture substrate of claim 25 wherein the entomopathogenic fungus is a genus selected from the group consisting of Beauveria, Metarhizium, Paecilomyces, Tolypocladium, Aspergillus, and Hirsutella.

27. The solid culture substrate of claim 26 wherein the entomopathogenic fungus is a species selected from the group consisting of Beauveria bassiana, Metarhizium flavoviride, Metarhizium anisopliae, Paecilomyces fumusoroseus, and Paecilomyces farinosus.

28. The substrate of claim 21 wherein the inert solid support is selected from the group consisting of corn cob woody fractions, plastic rings, diatomaceous earth, ceramic rings, ceramic saddles, stainless steel rings, crystalline silica, and clay.

29. The solid culture substrate comprising an extract of barley sorbed onto an inert solid support.

30. The solid culture substrate of claim 29 wherein the inert solid support is nutritionally inert.

31. The solid culture substrate of claim 29 wherein the solid culture substrate is in a form suitable for growing an entomopathogenic fungus.

32. The solid culture of claim 29 wherein the entomopathogenic fungus is a Deuteromycete fungus.

33. The solid culture substrate of claim 32 wherein the entomopathogenic fungus is a Hyphomycetes fungus.

34. The solid culture substrate of claim 33 wherein the entomopathogenic fungus is a genus selected from the group consisting of Beauveria, Metarhizium, Paecilomyces, Tolypocladium, Aspergillus, and Hirsutella.

35. The solid culture substrate of claim 34 wherein the entomopathogenic fungus is selected from the group consisting of Beauveria bassiana, Metarhizium flavoviride, Metarhizium anisopliae, Paecilomyces fumusoroseus, and Paecilomyces farinosus.

36. The solid culture substrate of claim 29 wherein the inert solid support is diatomaceous earth.

37. The solid culture substrate of claim 29 where the extract of barley is a water extract of barley.

38. A solid culture substrate comprising barley mixed with an inert material.

39. The solid culture substrate of claim 38 wherein the barley is selected from the group consisting of barley flakes, pearled barley, ground barley or barley extract.

40. The solid culture substrate of claim 38 wherein the solid culture substrate can grow an entomopathogenic fungus.

41. The solid culture of claim 38 wherein the entomopathogenic fungus is a Deuteromycete fungus.

42. The solid culture substrate of claim 41 wherein the entomopathogenic fungus is a Hyphomycetes fungus.

43. The solid culture of claim 42 wherein the entomopathogenic fungus is a genus selected from the group consisting of Beauveria, Metarhizium, Paecilomyces, Tolypocladium, Aspergillus, and Hirsutella.

44. The solid culture substrate of claim 43 wherein the entomopathogenic fungus is selected from the group consisting of Beauveria bassiana, Metarhizium flavoviride, Metarhizium anisopliae, Paecilomyces fumusoroseus, and Paecilomyces farinosus.

45. The solid culture substrate of claim 38 wherein the inert material is selected from the group consisting of straw, saw dust, wood chips, corn cob fractions, grain hulls, plastic rings, diatomaceous earth, ceramic rings, ceramic saddles, stainless steel rings, crystalline silica, and clay.

46. A packaged solid culture substrate comprising barley as a substrate and instructions for using the substrate to grow entomopathogenic fungus.

47. The packaged solid culture of claim 46 wherein the barley is selected from the group consisting of barley flakes, pearled barley, or ground barley or barley extract.

48. The packaged solid culture of claim 46 wherein the entomopathogenic fungus is a Deuteromycete fungus.

49. The solid culture substrate of claim 48 wherein the entomopathogenic fungus is a Hyphomycetes fungus.

50. The solid culture of claim 49 wherein the entomopathogenic fungus is a genus selected from the group consisting of Beauveria, Metarhizium, Paecilomyces, Tolypocladium, Aspergillus, and Hirsutella.

51. The solid culture of claim 50 wherein the entomopathogenic fungus is a species selected from the group consisting of Beauveria bassiana, Metarhizium flavoviride Metarhizium anisopliae, Paecilomyces fumusoroseus, and Paecilomyces farinosus.

52. A packaged solid culture substrate comprising processed barley as a substrate and instructions for using the substrate to grow fungi.

53. The packaged solid culture of claim 52 wherein the processed barley is selected from the group consisting of barley flakes, pearled barley, or ground barley or barley extract.

54. The packaged solid culture of claim 52 wherein the fungus is a Deuteromycete fungus.

55. A method of growing an entomopathogenic fungus comprising providing a barley substrate and combining the barley substrate with a culture of an entomopathogenic fungus under conditions which support growth of an entomopathogenic fungus.

56. A method of claim 55 wherein the barley substrate is selected from the group consisting of barley flakes, pearled barley. ground barley, or barley extract.

57. A method of claim 55 wherein the conidia of the entomopathogenic fungus are recovered.

58. The solid culture of claim 55 wherein the entomopathogenic fungus is a Deuteromycete fungus.

59. The solid culture substrate of claim 58 wherein the entomopathogenic fungus is a Hyphomycetes fungus.

60. The method of claim 59 wherein the entomopathogenic fungus is a genus selected from the group consisting of Beauveria, Metarhizium, Paecilomyces, Tolypocladium, Aspergillus, and Hirsutella.

61. A method of claim 60 wherein the entomopathogenic fungus is a species selected from the group consisting of Beauveria bassiana, Metarhizium flavoviride, Metarhizium anisopliae, Paecilomyces fumusoroseus, and Paecilomyces farinosus.

62. A method of growing a fungus comprising providing a processed barley substrate and combining the processed barley substrate with a culture of a fungus under conditions which support growth of a fungus.

63. The method of claim 62 wherein the processed barley substrate is selected from the group consisting of barley flakes, pearled barley, ground barley, or barely extract.

64. The method of claim 62 wherein the conidia of the fungus are recovered.

65. The method of claim 62 wherein the fungus is a Deuteromycete fungus.

66. The method of claim 62 wherein the conidia yield is significantly higher than the conidia yield of the fungus grown on a rice substrate under the same conditions.

67. The method of claim 62 wherein total conidia production of the fungus is 1×10¹³ per kilogram of processed barley substrate.

68. The method of claim 62 wherein the fungus is light dependent.

69. The method of claim 62 wherein the fungus conidiates in the absence of light.

70. The method of claim 69 wherein the fungus is Paecilomyces fumusoroseus.
